(19) United States
(12) Patent Application Publication
(10) Pub. No.: US 2023/0255778 A1
(43) Pub. Date: Aug. 17, 2023

(54) APPARATUS AND METHOD FOR INSERTING A CONDYLAR IMPLANT ASSEMBLY

(71) Applicant: DePuy Synthes Products, Inc., Raynham, MA (US)

(72) Inventor: Ross Hamel, West Chester, PA (US)

(21) Appl. No.: 17/670,898

(22) Filed: Feb. 14, 2022

(51) Int. Cl. A61F 2/38; A61F 2/46; A61M 29/02

(52) U.S. Cl. CPC A61F 2/3859; A61F 2/46; A61M 29/02; A61F 2002/3895; A61F 2250/0009

(57) ABSTRACT

The disclosure generally relates to condylar implant assemblies and methods for inserting an implant assembly for supporting the condylar head of a bone. More particularly the disclosure relates to an implant assembly for use in support of the condyle of a humerus, femur, or other bones having a condyle comprising spongy bone. In some implementations, the implant assembly may be inserted into the head of the bone and bands of a dilating member of the implant assembly may be expanded through slots in a tube of the implant assembly for supporting the condylar head of the bone.

### FIELD OF THE DISCLOSURE

The disclosure generally relates to condylar implant assemblies and methods for inserting an implant assembly for supporting the condylar head of a bone. More particularly the disclosure relates to an implant assembly for use in support of the condyle of a humerus, femur, or other bones having a condyle comprising spongy bone. In some implementations, the implant assembly may be inserted into the head of the bone and bands of a dilating member of the implant assembly may be expanded through slots in a tube of the implant assembly for supporting the condylar head of the bone.

### BACKGROUND

As can be appreciated by those familiar with the repair of bones with compromised bone structures, such as those affected by osteoporosis, providing support to affected bones is challenging. Compromised bone structures may be bone structures affected by, for example, osteoporosis or other bone weakening diseases, accidents, or birth defects. Current surgical solutions disclosed by the prior art include repairing broken bones via an open reduction method requiring screws, pins, rods, or a plate to hold a fragmented bone in place; a closed reduction method in which a cast or splint are used to position bones back together; the use of cement-like material injected into a broken or collapsed spinal bone to assist in strengthening the bone; and a balloon kyphoplasty apparatus in which a drill, special balloon, and cement-like material are used to correct an affected area of bone. However, these devices are prone to destabilization, such as when osteoporosis advances and the bone structure is further weakened.

To this end, there is a need for an improved device that provides stabilization for the condylar head of a bone, specifically, the head of the humerus, femur, or other bones that have compromised bone structure, such as due to osteoporosis.

### BRIEF DESCRIPTION OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale, or in schematic in the interest of clarity and conciseness. Not every component may be labeled in every drawing. Like reference numerals in the figures may represent and refer to the same or similar element or function. In the drawings:

FIG. 1 is a side, perspective view of an exemplary implant assembly in a first position in accordance with the present disclosure.

FIG. 2 is a side, perspective view of the implant assembly of FIG. 1 in a second position in accordance with the present disclosure.

FIG. 3A is a cross-sectional view of the implant assembly of FIG. 1 in the first position in accordance with the present disclosure.

FIG. 3B is a cross-sectional view of the implant assembly in the second position of FIG. 2 in accordance with the present disclosure.

FIG. 4 is a side, perspective view of an exemplary tube of the implant assembly of FIG. 1.

FIG. 5A is a side, perspective view of an exemplary dilating member of the implant assembly of FIG. 1 in a first position in accordance with the present disclosure.

FIG. 5B is a side, perspective view of an exemplary dilating member of the implant assembly of FIG. 2 in a second position.

FIG. 5C is a side, perspective view of another exemplary dilating member of the implant assembly of FIG. 2 in a second position.

FIG. 6 is a diagrammatic perspective view of a system for positioning an exemplary implant assembly within the humeral head of a patient constructed in accordance with the present disclosure.

FIG. 7 is a cross-sectional side view of an exemplary use of an implant assembly positioned within the humeral head in a second position in accordance with the present disclosure.

FIG. 8 is a side view of an exemplary bone plate positioned adjacent a humeral head of a patient having an exemplary implant assembly within the humeral head, in accordance with the present disclosure.

FIG. 9 is a cross-sectional view taken along lines 9-9 of FIG. 8 in conjunction with an exemplary implant assembly, in accordance with the present disclosure.

### SUMMARY OF THE INVENTION

Condylar implant assemblies and methods for inserting and/or removing an implant assembly for supporting the condylar head of a bone are described, including an implant assembly having a tube with a longitudinal length having two or more slots extending partially along the longitudinal length. The tube may have a first end having a first internal cross-sectional distance and a second end having a second internal cross-sectional distance smaller than the first internal cross-sectional distance such that an internal step is formed within the tube at the second end. The implant assembly may further comprise a driving device positioned within the first end of the tube; and a dilating member positioned between the driving device and the internal step within the tube. The dilating member may have a proximal end, a distal end, a longitudinal axis parallel to the longitudinal length of the tube, and a plurality of bands extending along the longitudinal axis between the proximal end and the distal end, each of the plurality of bands movable through a corresponding one of the two or more slots of the tube when the dilating member is compressed between the driving device and the internal step of the tube. The driving device 8 may be, for example, a set screw, a partially or completely serrated rod, and/or a rod-wedge assembly. For explanatory purposes, the term driving device may be used interchangeable with the set screw herein.

In some implementations, the two or more slots of the tube may be four or more slots of the tube. The two or more slots of the tube of the implant assembly may be positioned proximate to the second end of the tube.

In some implementations, the plurality of bands of the implant assembly may have a first position within the tube when the dilating member is extended and may have a second position extending through the two or more slots when the dilating member is compressed between the set screw and the internal step of the tube. The plurality of bands of the implant assembly in the second position may form an arc and/or a partial trapezoid. The dilating member of the implant assembly may be moveable between a first position between the set screw and the internal step within the tube and a second position between the set screw and the internal step within the tube. In some implementations, in the first position the plurality of bands is unextended and in the second position the plurality of bands extend through the corresponding one of the two or more slots of the tube when the dilating member is compressed between the set screw and the internal step of the tube.

In some implementations, the second end of the tube of the implant assembly may have external threads.

In some implementations, the implant assembly may have a bushing positioned between the set screw and the distal end of the dilating member. The plurality of bands of the implant assembly may have a first position when the dilating member is unextended and may have a second position when the plurality of bands is extending through the two or more slots when the dilating member is compressed between the bushing and the internal step of the tube.

Methods of making an implant assembly are described, including a method comprising inserting a dilating member into a tube having a longitudinal length, the tube having two or more slots extending partially along the longitudinal length, the tube having a first end having a first internal cross-sectional distance and a second end having a second internal cross-sectional distance smaller than the first internal cross-sectional distance such that an internal step is formed within the tube at the second end. The dilating member may have a proximal end, a distal end, a longitudinal axis parallel to the longitudinal length of the tube, and a plurality of bands extending along the longitudinal axis between the proximal end and the distal end. The method may further comprise inserting a set screw within the first end of the tube, such that the dilating member is between the set screw and the internal step of the tube, and advancing the set screw in the direction of the second end of the tube compresses the dilating member, thereby moving each of the plurality of bands through a corresponding one of the two or more slots of the tube.

In some implementations, methods of making an implant assembly may also include inserting a bushing between the set screw and the distal end of the dilating member. In some implementations, the plurality of bands may be movable through the two or more slots of the tube. The plurality of bands may also be moveable between a first position, when the dilating member is unextended, and a second position when the dilating member is compressed between the set screw and the internal step of the tube. The plurality of bands of the implant assembly in the second position may form an arc and/or a partial trapezoid.

Methods of positioning the implant assembly are disclosed, including a method comprising placing an implant assembly through an incision within a patient into a bone of the patient. The implant assembly may have a tube with a longitudinal length having two or more slots extending partially along the longitudinal length, the tube having a first end having a first internal cross-sectional distance and a second end having a second internal cross-sectional distance smaller than the first internal cross-sectional distance such that an internal step is formed within the tube at the second end. The implant assembly may comprise a set screw positioned within the first end of the tube; and a dilating member positioned between the set screw and the internal step within the tube, the dilating member having a proximal end, a distal end, a longitudinal axis parallel to the longitudinal length of the tube, and a plurality of bands extending along the longitudinal axis between the proximal end and the distal end. In some implementations, the implant assembly may be positioned through the incision within the patient into the bone of the patient through a plate positioned on the surface of the bone. The first end of the tube of the implant assembly may engage with the plate. In some implementations, the implant assembly may be positioned through the incision within the patient into an opening, such as a drilled hole, within the bone of the patient. In some implementations, the implant assembly may be positioned through the incision within the patient through a bone plate positioned on a surface of the bone through an opening within the bone of the patient where the opening within the bone of the patient is substantially aligned with one or more fastener holes of the plate. In some implementations, the first end of the tube of the implant assembly may engage with the one or more fastener holes of the bone plate, such as with threaded sides of the fastener holes.

In some implementations, the second end of the tube may be positioned internal to the bone of the patient. In some implementations, the method of positioning the implant assembly may include engaging a surgical screwdriver with the set screw such that the set screw is advanced within the tube toward the second end, thereby compressing the dilating member between the set screw and an internal step of the second end of the tube until each of the plurality of bands of the dilating member move outwardly through corresponding ones of the two or more slots of the tube. The method of positioning the implant assembly may also include moving the set screw within the tube toward the first end until each of the plurality of bands of the dilating member move inwardly through the corresponding ones of the two or more slots of the tube, such that the dilating member is unextended, thereby allowing for the removal of the implant assembly from the bone.

In some implementations, the implant assembly may also have a bushing between the set screw and the dilating member in the tube. In some implementations, the method of positioning the implant assembly may include each of the plurality of bands forming one or more of an arc and a partial trapezoid when the plurality of bands of the dilating member are moved outwardly through corresponding ones of the two or more slots of the tube.

### DETAILED DESCRIPTION

Before explaining at least some implementations of the present disclosure in detail by way of exemplary language and results, it is to be understood that the present disclosure is not limited in its application to the details of construction and the arrangement of the components set forth in the following description. The present disclosure is capable of other implementations or of being practiced or carried out in various ways. As such, the language used herein is intended to be given the broadest possible scope and meaning; and the implementations are meant to be exemplary, not exhaustive. Also, it is to be understood that the phraseology and terminology employed herein is for the purpose of description and should not be regarded as limiting.

Unless otherwise defined herein, scientific, and technical terms used in connection with the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the medical procedures and techniques of surgery, anesthesia, orthopedics, and trauma repair described herein are those well-known and commonly used in the art. Standard techniques are used for diagnostic and therapeutic applications of the present disclosure.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

All the articles, compositions, kits, and/or methods disclosed herein can be made and executed without undue experimentation in light of the present disclosure. While the articles, compositions, kits, and/or methods have been described in terms of particular implementations, it will be apparent to those of skill in the art that variations may be applied to the articles, compositions, kits, and/or methods and in the steps or in the sequence of steps of the methods described herein without departing from the concept, spirit, and scope of the present disclosure. All such similar substitutes and modifications apparent to those skilled in the art are deemed to be within the spirit, scope, and concept of the present disclosure as defined by the appended claims.

As utilized in accordance with the present disclosure, the following terms, unless otherwise indicated, shall be understood to have the following meanings: the use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “some implementations,” “an embodiment,” “some embodiments,” “one example,” “for example,” or “an example” means that a particular element, feature, structure, or characteristic described in connection with the embodiment is included in at least some implementations. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the terms “about” and “approximately” are used to indicate that a value includes the inherent variation of error for a composition/apparatus/device, the method being employed to determine the value, or the variation that exists among the study subjects. For example, but not by way of limitation, when the terms “about” or “approximately” are utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. The term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties (or assemblies, mechanisms, or components and the like) to one another as well as indirect association/binding of two moieties (or assemblies, mechanisms, or components and the like) to one another. Non-limiting examples of associations/couplings include covalent binding of one moiety (or assemblies, mechanisms, or components and the like) to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example. Additionally, physical couplings and/or mechanical couplings could be used for the direct or indirect association/binding of two moieties (or assemblies, mechanisms, or components and the like) to one another.

As used herein, the term “patient” or “subject” is meant to include all organisms, whether alive or dead, including any species having soft tissues and bones. For example, a method according to the inventive concepts disclosed herein may be used within a living/dead human, horse, cow, sheep, cat, dog, and the like.

In accordance with the present disclosure, the general purpose of the disclosure there is a need for an improved implant that provides stabilization for the condylar head of a bone. Specifically, the head of the humerus, femur, or other bones that have compromised bone structure, as due to osteoporosis.

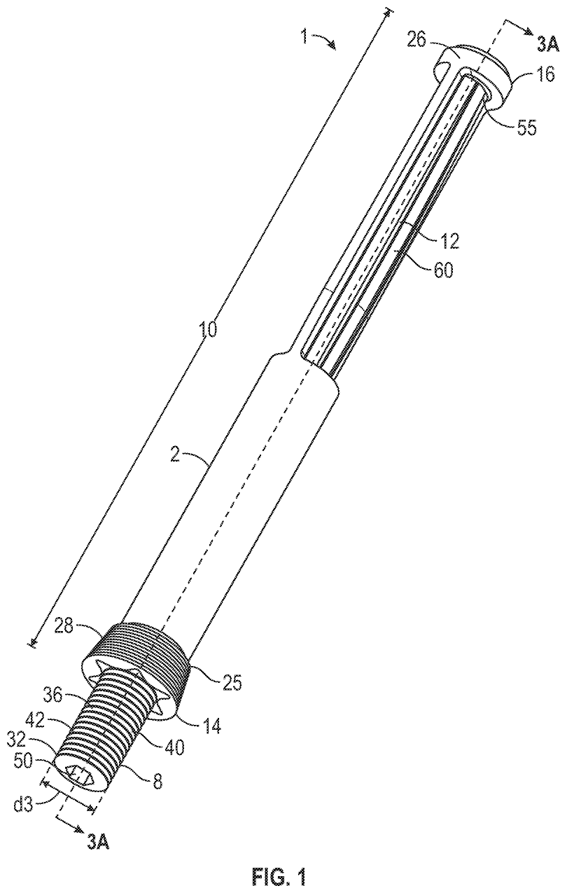

Referring now to the drawings, FIG. 1 illustrates a side, perspective view of an exemplary implant assembly 1 in a first position in accordance with the present disclosure. The implant assembly 1 may comprise a tube 2 having a first end 14 and a second end 16, a dilating member 4 positioned within the tube 2 between the first end 14 and the second end 16, and a driving device 8 positioned at least partially within the tube 2 between the dilating member 4 and the second end 16. The driving device 8 may be, for example, a set screw, a partially or completely serrated rod, and/or a rod-wedge assembly. For explanatory purposes, the term driving device 8 may be used interchangeable with the set screw 8 herein.

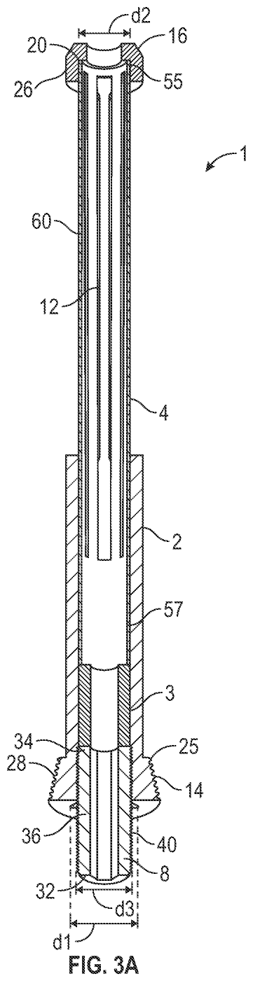

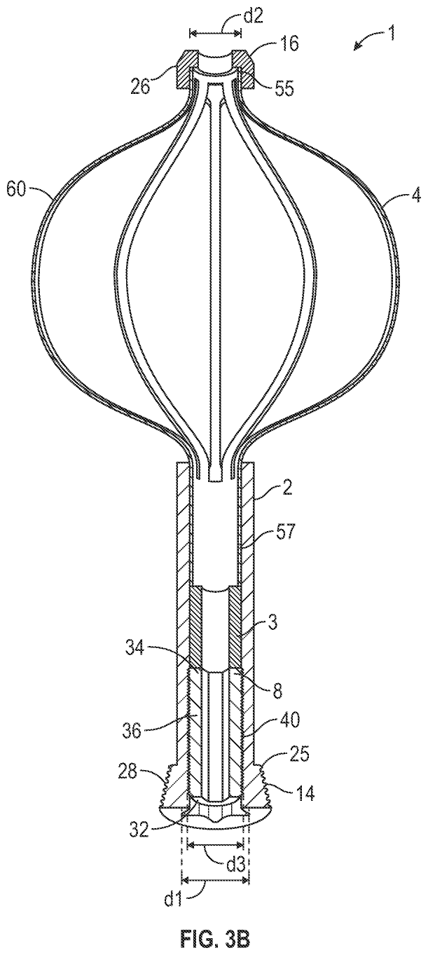

In some of the presently disclosed non-limiting implementations, the implant assembly 1 may also comprise a bushing 3 (FIG. 3A) positioned in the tube 2 between the set screw 8 and the dilating member 4. In some non-limiting implementations, the bushing may be constructed of any type of biocompatible material, such as stainless steel, titanium, biocompatible polymers, and combinations of the same.

As shown in FIGS. 1-4, the tube 2 may have a longitudinal length 10 extending between the first end 14 and the second end 16, and two or more slots 12 extending partially along the longitudinal length 10. In some of the presently disclosed non-limiting implementations, the two or more slots 12 of the tube 2 are four or more slots 12. In some implementations, as seen in FIG. 1, the two or more slots 12 are positioned proximate to the second end 16 of the tube 2. The two or more slots 12 may extend from the second end 16 to approximately the first end 14. The two or more slots 12 may extend from the second end 16 to approximately the center of the tube 2. The two or more slots 12 may extend more than half of the length of the tube 2. Each of the slots 12 may have a width and a length.

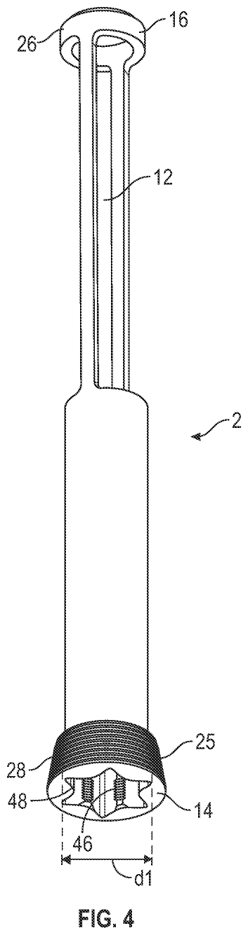

In some non-limiting implementations, as illustrated in the cross-sectional view of FIG. 3A, the first end 14 of the tube 2 may have a first internal cross-sectional diameter d1, and the second end 16 of the tube 2 may have a second internal cross-sectional diameter d2 smaller than the first internal cross-sectional diameter d1 such that an internal step 20 is formed within the tube 2 at the second end 16. In some implementations, the tube 2 may have an internal surface 48 and a plurality of threads 46 formed on the internal surface 48 (FIG. 4) proximate to the first end 14 of the tube 2.

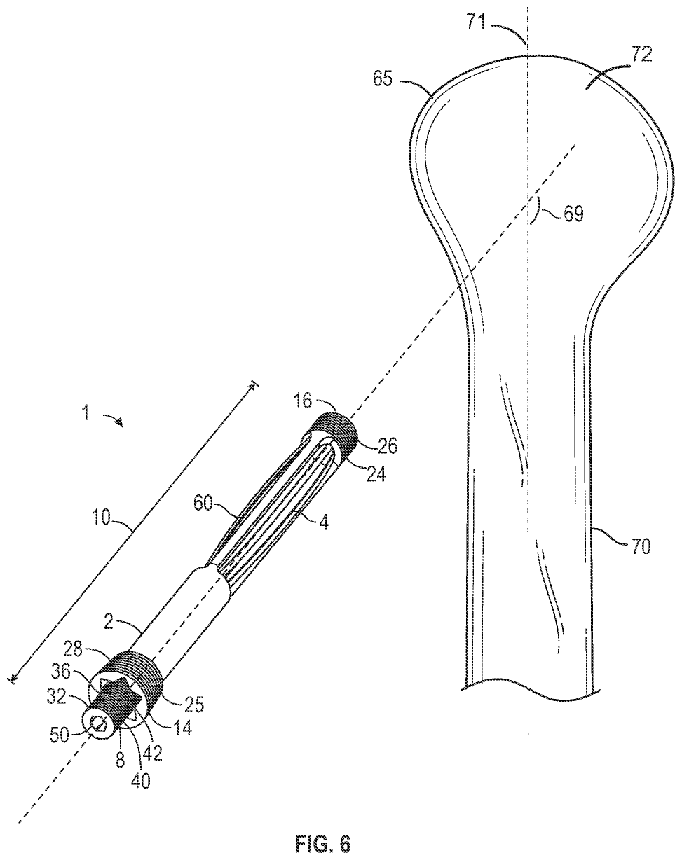

In some implementations, the second end 16 of the tube 2 has external threads 24 (FIG. 6). In some implementations, an external surface 25 of the first end 14 of the tube 2 has external threads 28. The tube 2 may be constructed of any type of biocompatible material, such as stainless steel, titanium, biocompatible polymers, and combinations of the same.

In some of the presently disclosed non-limiting implementations, the second end 16 of the tube 2 is rounded. In some implementations, the second end 16 of the tube 2 may have a semi-circular shape engageable with an interior of a bone. In some implementations, the second end 16 of the tube 2 may have teeth or ridges engageable with an interior of a bone.

In some implementations, as shown in FIGS. 3A-3B, the set screw 8 may comprise a first end 32, a second end 34, and a shaft 36 extending between the first end 32 and the second end 34. The shaft 36 of the set screw 8 has a third diameter d3 and an outer surface 40. In some implementations, the outer surface of the shaft 36 has a plurality of threads 42. In some implementations, the plurality of threads 42 of the outer surface 40 of the shaft 36 of the set screw 8 are engageable with the plurality of threads 46 on the internal surface 48 of the first end 14 of the tube 2. The set screw 8 may have a first position within the first end 14 of the tube 2 and the set screw 8 may be advanced to a second position within the first end 14 of the tube 2 toward the second end 16 of the tube 2. In some implementations, the first end 32 of the set screw 8 has a socket 50 configured engageable with a surgical screwdriver. The set screw 8 may be constructed of stainless steel, titanium, biocompatible polymers, and combinations of the same.

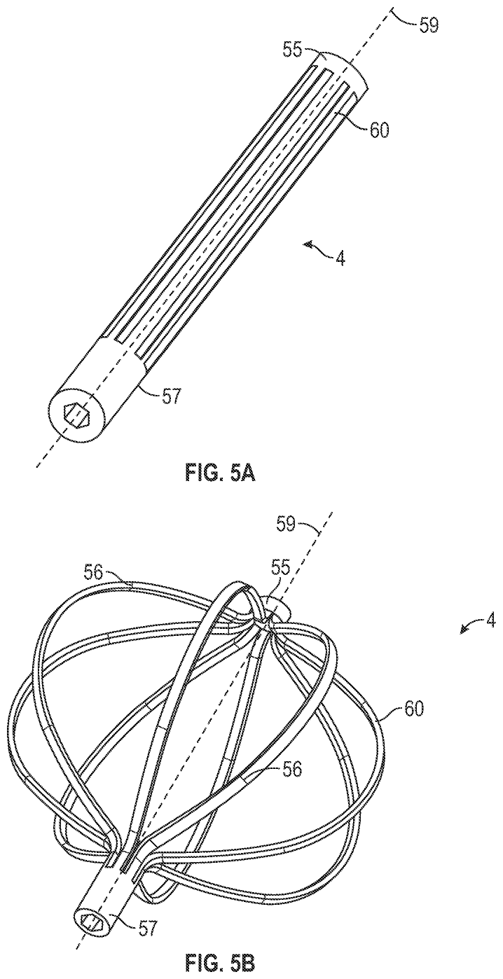

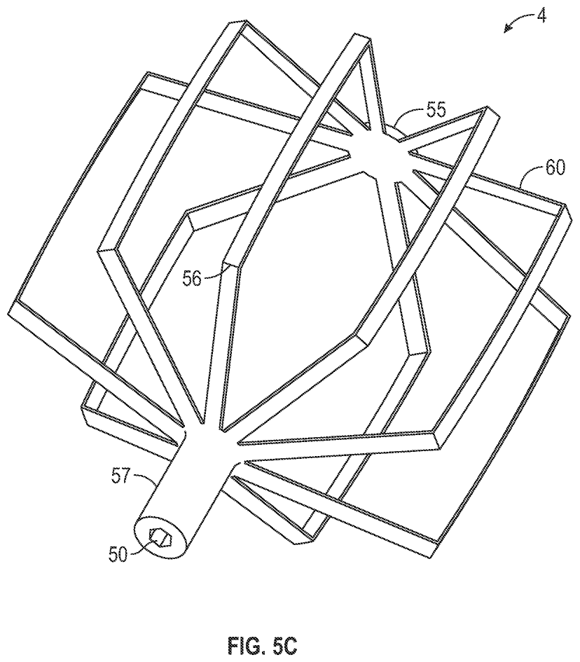

As shown in FIG. 5A, the dilating member 4 has a proximal end 55, a distal end 57, a longitudinal axis 59 parallel to the longitudinal length 10 of the tube 2 when the dilating member 4 is positioned within the tube 2, and a plurality of bands 60 extending along the longitudinal axis 59 between the proximal end 55 and the distal end 57. In some implementations, the bands 60 may extend substantially the length of the dilating member between the proximal end 55 and the distal end 57. In some implementations, the bands 60 may be positioned proximate to the proximal end 55 of the dilating member 4 and may extend toward the distal end 57 at least half way along the length of the dilating member 4. In some implementations, each of the plurality of bands 60 may have a width which may be sized to be smaller than the width of the slots 12 of the tube 2.

The plurality of bands 60 of the dilating member 4, and/or the dilating member 4 as a whole, may be constructed of a flexible material. In some implementations, the bands 60 and/or one or more portions of the dilating member 4 may be constructed of super elastic, shape memory nitinol, or the like.

In some implementations, the plurality of bands 60 may comprise a same number of bands 60 as a number of the slots 12. For example, the dilating member 4 may have two or more bands 60. In some implementations, the dilating member 4 may have eight bands 60 and the tube may have eight corresponding slots 12. In some implementations, the dilating member 4 may have more bands 60 than the tube 2 has slots 12.

As shown in FIG. 5A, the plurality of bands 60 may have a first position when the dilating member 4 is extended. As show in FIG. 5B-5C, the plurality of bands 60 may have a second position when the dilating member 4 is compressed. Each of bands of the plurality of bands 60 may be positioned symmetrically or asymmetrically around the longitudinal axis 59 of the dilating member 4. The plurality of bands 60 may be constructed of a material so as to be movable outwardly from the first position to the second position, and movable inwardly from the second position to the first position. The form of the second position may be preset 56 in the material such that when the dilating member 4 is compressed, the plurality of bands 60 of the dilating member 4 form a preset shape. For example, the material may be deformed past an elastic deformation point of the material, or otherwise manufactured, to form the preset shape, such that the plurality of bands 60 return to the preset shape when the plurality of bands 60 are in the second position. In some implementations, one or more of the bands 60 of the plurality of bands 60 in the second position form an arc (FIG. 5B). In some implementations, one or more of the bands 60 of the plurality of bands 60 in the second position form a partial trapezoid (FIG. 5C).

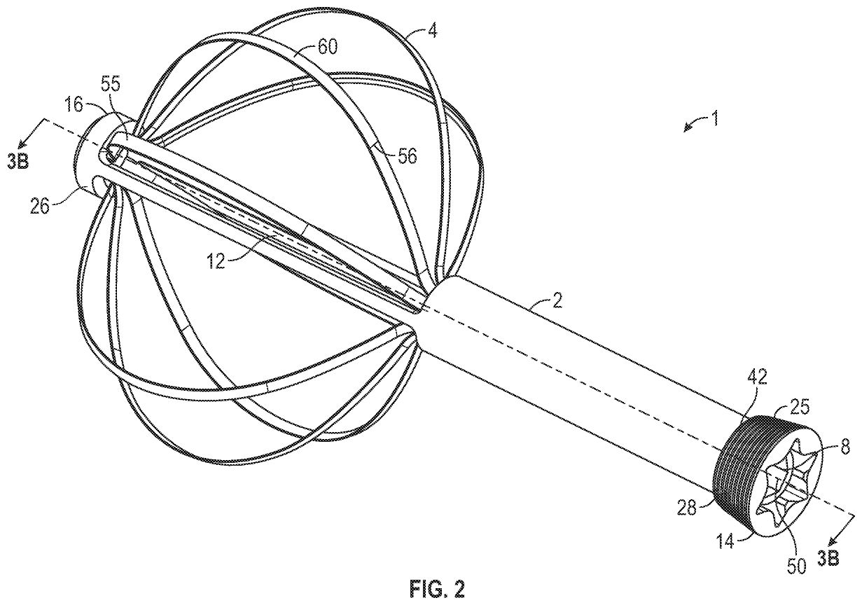

Returning now to FIGS. 1-3B, the implant assembly 1 may be changeable between a first state and a second state. In the first state (FIGS. 1 and 3A), the dilating member 4 may be extended within the tube 2 between the set screw 8 and the second end 16 of the tube 2 such that the plurality of bands 60 are within the tube 2. In the second state (FIGS. 2 and 3B), the dilating member 4 may be extended within the tube 2 between the set screw 8 and the internal step 20 of the second end 16 of the tube 2 such that each of the plurality of bands 60 are extended and positioned through a corresponding one of the two or more slots 12 of the tube 2. In some implementations, the bushing 3 is positioned in the first end 14 of the tube 2 between the distal end 57 of the dilating member 4 and the first end 32 of the set screw 8 such that rotation from the set screw 8 is not translated to the dilating member 4. In such implementations, each of the plurality of bands 60 are extended and positioned through a corresponding one or the two or more slots 12 of the tube 2 when the dilating member 4 is compressed between the bushing 3 and the internal step 20 of the second end 16 of the tube 2.

It will be understood that the implant assembly 1 may have interim states between the first state and the second state in which the bands 60 are moved through the corresponding slots 12, and that the position of the set screw 8 may be used to control an outward position of and/or the amount of extension of the plurality of bands 60 in relation to the slots 12 of the tube 2.

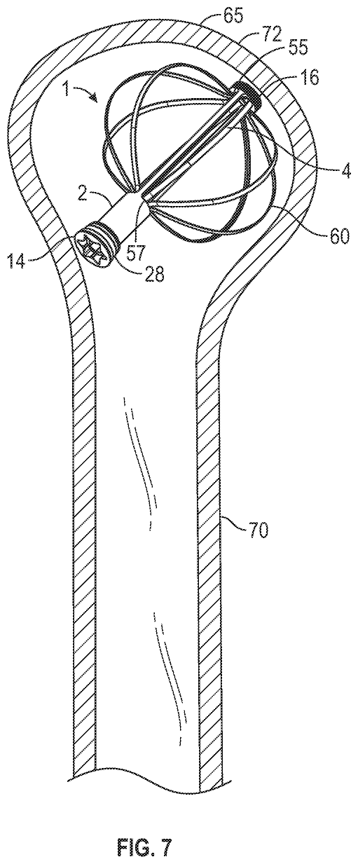

It will further be understood that the internal step 20 may be replaced by an end cap or other component that stops longitudinal movement of the dilating member 4 within the tube 2. As shown in FIGS. 6 and 7, the implant assembly 1 may be inserted into the head 65 of the humerus 70 of a patient to provide support. Although the system will be described herein by way of example for assisting in the support and stabilization of the head 65 of the humerus 70, it should be understood that the system may be used for support and stabilization within various bones of the patient. For this reason, the implant assembly 1 may be constructed in various sizes. In use, a surgeon may guide the implant assembly 1 through an incision in the skin of the patient. In some implementations, the surgeon may guide the implant assembly 1 through an incision in the patient's skin at an angle 69 in relation to a longitudinal axis 71 of the humeral head 65 of the humerus 70, as shown in FIG. 6. In one implementation, the angle of insertion 69 of the implant assembly 1 in relation to the longitudinal axis 71 of the humeral head 65 may be between approximately 90° to approximately 150°. In one implementation, the angle of insertion 69 of the implant assembly 1 is approximately 135°. In some implementations, the implant assembly 1 may be positioned through the incision within the patient through an opening within the bone of the patient. The opening within the bone of the patient may be a drill hole or the like. The external threads 24 of the second end 16 of the tube 2 may engage the opening within the bone of the patent. The implant assembly 1 is not shown to scale with the humeral head 65 in FIG. 6, for explanatory purposes.

The implant assembly 1 may pass through the surface 72 of the humerus 70 until the implant assembly 1 is positioned within the head 65 of the humerus 70 of the patient. In this position, a surgical screwdriver may engage the first end 32 of the set screw 8 in order to rotate the set screw 8 in a first rotating direction to advance the set screw 8 within the tube 2 toward the second end 16 of the tube 2, transitioning the set screw 8 from the first position to the second position within the first end 14 of the tube 2 (FIGS. 1-3B). The dilating member 4 is then compressed between the internal step 20 of the second end 16 of the tube 2 and the set screw 8 until each of the plurality of bands 60 of the dilating member 4 move through corresponding ones of the two or more slots 12 of the tube 2, as shown in FIG. 7. Though the plurality of bands 60 in FIG. 7 is shown as filling a portion of the interior of the humeral head 65, the plurality of bands 60 may be larger or smaller and may fill more or less of the area within the humeral head 65.

In some implementations, the bushing 3 is positioned between the second end 34 of the set screw 8 and the distal end 57 of the dilating member 4 such that when the set screw 8 is rotated in the first rotating direction, the set screw 8 is advanced toward the second end 16 of the tube 2 transitioning the set screw 8 from the first position to the second position within the first end 14 of the tube 2 and advancing the bushing 3, without translating the rotation of the set screw 8 to the dilating member 4. The dilating member 4 is then compressed between the internal step 20 of the second end 16 of the tube 2 and the bushing 3 until each of the plurality of bands 60 of the dilating member 4 move through corresponding ones of the two or more slots 12 of the tube 2.

The surgical screwdriver may then be disengaged from the first end 32 of the set screw 8 and removed from the surgical field. In another implementation, the surgical screwdriver engages the socket 50 in the first end 32 of the set screw 8.

In some implementations, the implant assembly 1 may be removed from the head 65 of the humerus 70 of the patient. The surgical screwdriver may engage the first end 32 of the set screw 8 in order to rotate the set screw 8 in a second rotating direction to move the set screw 8 within the tube 2 toward the first end 14 of the tube 2, transitioning the set screw 8 from the second position to the first position within the first end 14 of the tube 2 (FIGS. 1-3B), until each of the plurality of bands 60 of the dilating member 4 move inwardly through the corresponding ones of the two or more slots 12 of the tube 2, such that the dilating member 4 is unextended. The implant assembly 1 may then be removed from the bone.

FIG. 7 is a cross-sectional side view of an exemplary use of the implant assembly 1 positioned within the head 65 of the humerus 70 in a second position in accordance with the present disclosure. The second position of the dilating member 4 may be adjusted by the surgeon to permit optimal support and stabilization of the bone.

In some cases, the surgical screwdriver may engage the first end 32 of the set screw 8 and may be rotated in a second rotating direction, such that the second rotating direction is opposite the first rotating direction. The rotation of the set screw 8 in the second rotating direction results in the repositioning of the set screw 8 in the direction of the first end 14 of the tube 2, from the second position of the set screw 8 to the first position of the set screw 8, causing the plurality of bands 60 of the dilating member 4 to move from the second position to the first position within the tube 2 for removal from the bone.

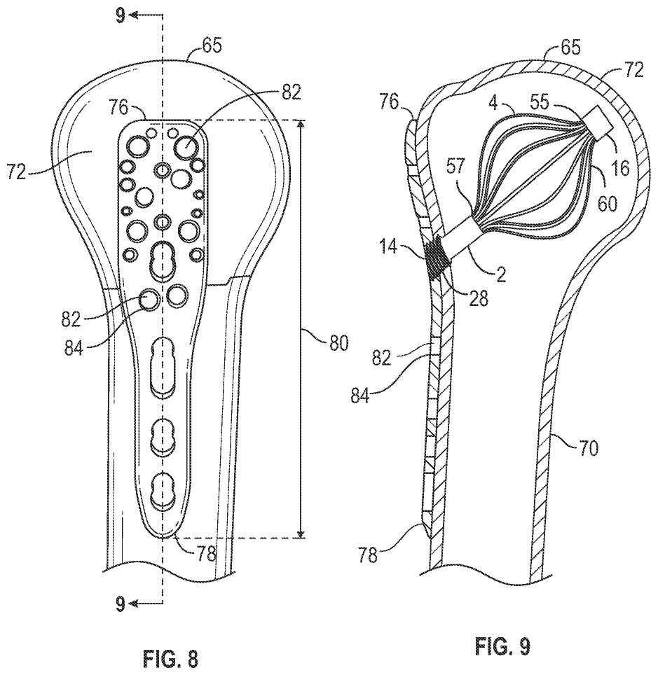

It will be appreciated by those of ordinary skill in the art that in some implementations, the implant assembly 1 may be used with one or more bone plates 74, an example of which is illustrated in FIGS. 8 and 9. The one or more bone plate 74 may be a plate that bridges one or more fractures in a bone 70 and holds each part of the fracture in alignment during healing. The bone plate 74 may have a first end 76, a second end 78, a length 80 which extends between the first end 76 and the second end 78, and one or more fastener holes 82. The one or more fastener hole 82 may be positioned on the length 80 between the first end 76 and the second end 78 of the bone plate 74. The bone plate 74 may be positioned adjacent the bone 70. The bone plate 74 may be constructed of, for example, stainless steel, titanium, biocompatible polymers, and combinations of the same.

In some implementations, the second end 16 or the first end 14 of the tube 2 of the implant assembly 1 may be engaged with the bone plate 74, such as with the one or more fastener hole 82 of the bone plate 74. In some implementations, the fastener hole 82 may have a threaded internal surface 84 and the external surface 26 of the second end 16 or the first end 14 of the tube 2 of the implant assembly 1 may have external threads 24 (FIG. 6) engageable with the threaded internal surface 84 of the fastener hole 82 of the bone plate 74.

As shown in FIG. 9, in some implementations, positioning the implant assembly 1 may include placing the implant assembly 1 through the incision within the patient through the bone plate 74 positioned on the surface 72 of the bone 70, and engaging the first end 14 of the tube 2 of the implant assembly 1 with the bone plate 74. In one implementation, the external threads 28 positioned on the external surface 25 of the first end 14 of the tube 2 may engage the threaded internal surface 84 of the one or more fastener hole 82, thereby fastening the implant assembly 1 to the one or more fastener hole 82 of the bone plate 74.

In some implementations, the first end 14 of the tube 2 of the implant assembly 1 may have an external locking element positioned on the external surface 25 of the first end 14. The internal surface 84 of the one or more fastener hole 82 may have an internal locking element such that when the implant assembly 1 is positioned through the one or more fastener hole 82 the external locking element of the first end 14 of the tube 2 engages the internal locking element of the one or more fastener hole 82 thereby attaching the implant assembly 1 to the bone plate 74.

In some implementations, positioning the implant assembly 1 may include placing the implant assembly 1 through the incision within the patient through the bone plate 74 positioned on the surface 72 of the bone 70 through an opening within the bone 70 of the patient wherein the opening within the bone 70 of the patient is substantially aligned with the one or more fastener hole 82 of the bone plate 74. The implant assembly 1 being engaged with the bone plate 74 facilitates stabilization and repairing of the bone 70.

An exemplary method of making the implant assembly 1 may comprise forming the tube 2 with the longitudinal length 10 having two or more slots 12 extending partially along the longitudinal length 10, the tube 2 having the first end 14 having the first internal cross-sectional diameter d1 and the second end 16 having a second internal cross-sectional diameter d2 smaller than the first internal cross-sectional diameter d1 such that an internal step 20 is formed within the tube 2 at the second end 16. In some of the presently disclosed non-limiting implementations, forming the tube 2 is defined further as providing the two or more slots 12 positioned proximate to the second end 16 of the tube 2. In some implementations, forming the tube 2 is defined further as providing four or more slots 12 in the tube 2.

In some of the presently disclosed non-limiting implementations, forming the set screw 8 is defined as forming the first end 32, the second end 34, and the shaft 36 extending between the first end 32 and the second end 34. The shaft 36 of the set screw 8 having the third cross-sectional diameter d3 and the outer surface 40. In some implementations, the outer surface of the shaft 36 has the plurality of threads 42. The third diameter d3 of the set screw 8 is less than the first cross-sectional diameter d1 of the first end 14 of the tube 2 such that the set screw 8 is positioned within the first end 14 of the tube 2. The set screw 8 may be constructed of stainless steel, titanium, biocompatible polymers, and combinations of the same.

In some of the presently disclosed non-limiting implementations, forming the implant assembly 1 further comprises forming a bushing 3. In some implementations, the bushing 3 is positioned between the set screw 8 and the distal end 57 of the dilating member 4 within the first end 14 of the tube 2. The bushing 3 may be constructed of stainless steel, titanium, biocompatible polymers, and combinations of the same.

In some of the presently disclosed non-limiting implementations, forming the dilating member 4 is defined further as forming the proximal end 55, the distal end 57, the longitudinal axis 59 parallel to the longitudinal length 10 of the tube 2, and the plurality of bands 60 extending along the longitudinal axis 59 between the proximal end 55 and the distal end 57, each of the plurality of bands 60 movable through a corresponding one of the two or more slots 12 of the tube 2 when the dilating member 4 is compressed between the set screw 8 and the internal step 20 of the tube 2. In some implementations, the dilating member 4 is compressed between the bushing 3 and the internal step 20 of the tube 2. The form of the second position can be formed as a preset 56 in the material such that when the dilating member 4 is compressed, the plurality of bands 60 of the dilating member 4 will form a preset shape. The dilating member 4 may be constructed of super elastic, shape memory nitinol, or the like.

In some of the presently disclosed non-limiting implementations, forming the dilating member 4 is defined further as forming the plurality of bands 60 to be movable through the two or more slots 12 of the tube 2 having a first position when the dilating member 4 is unextended and having a second position when the dilating member 4 is compressed between the set screw 8 and the internal step 20 of the second end 16 of the tube 2. In some implementations, the dilating member 4 is defined further to be formed to have a second position when the dilating member 4 is compressed between the bushing 3 and the internal step 20 of the second end 16 of the tube 2. In some implementations, the dilating member 4 is defined further to form an arc when each of the plurality of bands 60 of the dilating member 4 are in the second position. In some implementations, the dilating member 4 is defined further to form a partial trapezoid when each of the plurality of bands 60 of the dilating member 4 are in the second position. It is understood to those of ordinary skill in the art that each of bands of the plurality of bands 60 may be formed to be positioned asymmetrically around the longitudinal axis 59 of the dilating member 4. The plurality of bands 60 may be constructed of a material so as to be movable outwardly from the first position to the second position and movable inwardly from the second position to the first position. The dilating member 4 may be constructed of super elastic, shape memory nitinol, or the like.

In some of the presently disclosed non-limiting implementations, the external surface 26 of the second end 16 of the tube 2 may be formed to having external threads 24. In other implementations, the second end 16 of the tube 2 may be formed to be rounded (see FIG. 4).

In some of the presently disclosed non-limiting implementations, forming the first end 32 of the set screw 8 is defined further as having a socket 50 configured to engage the surgical screwdriver.

Forming the implant assembly 1 may include the creation and assembly of a plurality of components which could be created through various forms of metal forming such as cold sizing, extrusion, drawing, forging, powder metallurgy, friction drilling, rolling, and burnishing.

The components may be joined by welding, brazing, soldering, or other processes known to those of ordinary skill in the art. The implant assembly 1 may also be formed as a unitary structure, and the plurality of elongated members cut using an electrical discharge machining (EDM) process. The dilating member 4 of the implant assembly 1 may be created by the initial creation of a tube 2 having common dimensions. The various processes for forming the tube 2 may include but are not limited to turning, gun drilling/deep drilling, or other methods known to those of ordinary skill in the art. The plurality of elongated members of the second region can be formed by cutting the slots 12 using any suitable medium, such as a laser, a wire electrical discharge machining process, a water jet, plasma, milling, or sheering, or other methods known to persons of ordinary skill in the art. In some implementations, the tube 2 can be formed using a three-dimensional printing process.

The foregoing description provides illustration and description but is not intended to be exhaustive or to limit the inventive concepts to the precise form disclosed. Modifications and variations are possible in light of the above teachings or may be acquired from practice of the methodologies set forth in the present disclosure. Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set. No element, act, or instruction used in the present application should be construed as critical or essential to the invention unless explicitly described as such outside of the preferred implementation. Further, the phrase “based on” is intended to mean “based, at least in part, on” unless explicitly stated otherwise.

What is claimed is:

1. An implant assembly, comprising:
a tube with a longitudinal length having two or more slots extending partially along the longitudinal length, the tube having a first end having a first internal cross-sectional distance and a second end having a second internal cross-sectional distance smaller than the first internal cross-sectional distance such that an internal step is formed within the tube at the second end;
a driving device positioned within the first end of the tube; and
a dilating member positioned between the driving device and the internal step within the tube, the dilating member having a proximal end, a distal end, a longitudinal axis parallel to the longitudinal length of the tube, and a plurality of bands extending along the longitudinal axis between the proximal end and the distal end, each of the plurality of bands movable through a corresponding one of the two or more slots of the tube when the dilating member is compressed between the driving device and the internal step of the tube.

2. The implant assembly of claim 1, wherein the two or more slots of the tube are four or more slots.

3. The implant assembly of claim 1, wherein the two or more slots of the tube are positioned proximate to the second end of the tube.

4. The implant assembly of claim 1, wherein the plurality of bands has a first position within the tube when the dilating member is extended and have a second position extending through the two or more slots when the dilating member is compressed between the driving device and the internal step of the tube.

5. The implant assembly of claim 4, wherein each of the plurality of bands in the second position form one or more of an arc and a partial trapezoid.

6. The implant assembly of claim 1, wherein the dilating member is moveable between a first position between the driving device and the internal step within the tube and a second position between the driving device and the internal step within the tube, wherein in the first position, the plurality of bands are unextended, and wherein in the second position the plurality of bands extend through the corresponding one of the two or more slots of the tube when the dilating member is compressed between the driving device and the internal step of the tube.

7. The implant assembly of claim 1, wherein the second end of the tube has external threads.

8. The implant assembly of claim 1, further comprising a bushing positioned between the driving device and the distal end of the dilating member.

9. The implant assembly of claim 8, wherein the plurality of bands has a first position within the tube when the dilating member is unextended and have a second position wherein the plurality of bands extends through corresponding ones of the two or more slots of the tube when the dilating member is compressed between the bushing and the internal step of the tube.

10. A method of making an implant assembly, comprising:
inserting a dilating member in a tube with a longitudinal length, the tube having two or more slots extending partially along the longitudinal length, the tube having a first end having a first internal cross-sectional distance and a second end having a second internal cross-sectional distance smaller than the first internal cross-sectional distance such that an internal step is formed within the tube at the second end; the dilating member having a proximal end, a distal end, a longitudinal axis parallel to the longitudinal length of the tube, and a plurality of bands extending along the longitudinal axis between the proximal end and the distal end; and
inserting a driving device within the first end of the tube, such that the dilating member is between the driving device and the internal step of the tube, such that advancing the driving device compresses the dilating member, thereby moving each of the plurality of bands through a corresponding one of the two or more slots of the tube.

11. The method of claim 10, further comprising:
inserting a bushing between the driving device and the distal end of the dilating member.

12. The method of claim 10, wherein the two or more slots of the tube are four or more slots.

13. The method of claim 10, wherein the two or more slots of the tube are positioned proximate to the second end of the tube.

14. The method of claim 10, wherein the plurality of bands is movable through the two or more slots of the tube, and wherein the plurality of bands is moveable between a first position when the dilating member is unextended and have a second position when the dilating member is compressed between the driving device and the internal step of the tube.

15. The method of claim 14, wherein each of the plurality of bands in the second position form one or more of an arc and a partial trapezoid when the plurality of bands of the dilating member move outwardly through corresponding ones of the two or more slots of the tube.

16. The method of claim 10, wherein the second end of the tube has external threads.

17. A method of positioning an implant assembly, comprising:
placing the implant assembly through an incision within a patient into a bone of the patient, the implant assembly comprising:
a tube with a longitudinal length having two or more slots extending partially along the longitudinal length, the tube having a first end having a first internal cross-sectional distance and a second end having a second internal cross-sectional distance smaller than the first internal cross-sectional distance such that an internal step is formed within the tube at the second end;
a driving device positioned within the first end of the tube; and
a dilating member positioned between the driving device and the internal step within the tube, the dilating member having a proximal end, a distal end, a longitudinal axis parallel to the longitudinal length of the tube, and a plurality of bands extending along the longitudinal axis between the proximal end and the distal end;
wherein the second end of the tube is positioned in the bone of the patient;
engaging a surgical screwdriver with the driving device; and
advancing the driving device within the tube toward the second end such that the dilating member is compressed between the driving device and an internal step of the second end of the tube until each of the plurality of bands of the dilating member move outwardly through corresponding ones of the two or more slots of the tube.

18. The method of positioning the implant assembly of claim 17, further comprising:
moving the driving device within the tube toward the first end of the tube until each of the plurality of bands of the dilating member move inwardly through the corresponding ones of the two or more slots of the tube, such that the dilating member is unextended; and
removing the implant assembly from the bone.

19. The method of positioning the implant assembly of claim 18, wherein the implant assembly further comprises a bushing between the driving device and the dilating member in the tube.

20. The method of positioning the implant assembly of claim 17, wherein placing the implant assembly through the incision within the patient into the bone of the patient comprises placing the implant assembly through a bone plate positioned on a surface of the bone and engaging the first end of the tube of the implant assembly with the bone plate.
